(19) United States
(12) Patent Application Publication
(10) Pub. No.: US 2022/0280398 A1
(43) Pub. Date: Sep. 8, 2022

(54) METHOD FOR MANUFACTURING MICROCAPSULES CONTAINING A LIPOPHILIC ACTIVE INGREDIENT, MICROCAPSULES PREPARED BY SAID METHOD AND THE USE THEREOF

(71) Applicant: MICROCAPSULES TECHNOLOGIES, PUISEAUX (FR)

(72) Inventor: Gérard HABAR, BOURRON-MARLOTTE (FR)

(21) Appl. No.: 17/632,483

(22) PCT Filed: Jul. 20, 2020
(86) PCT No.: PCT/FR2020/051302

§ 371 Date: Feb. 2, 2022

(30) Foreign Application Priority Data
Aug. 6, 2019 (FR) 19 08996

(51) Int. Cl. A61K 8/11; A61K 8/73; A61K 8/81; A61K 8/891; A61Q 19/10; A61Q 5/12; A61Q 5/02; B01J 13/10; B01J 13/16; C11D 3/50; C11D 3/00; C11D 11/00

(52) U.S. Cl. CPC A61K 8/11; A61K 8/731; A61K 8/8147; A61K 8/891; A61Q 19/10; A61Q 5/12; A61Q 5/02; B01J 13/10; B01J 13/16; C11D 3/505; C11D 3/001; C11D 11/0017

(57) ABSTRACT

The present invention relates to a process for manufacturing reservoir-type microcapsules containing a lipophilic active agent, the wall of which comprises at least two polymers obtained by co-crosslinking of a polymer obtained by complex coacervation and of a copolymer of silicone, melamine-carbamate and polyurethane. The microcapsules thus prepared may be used in formulations containing surfactants, such as washing products or detergents, or in cosmetic formulations such as shampoos or soaps.

### FIELD OF THE INVENTION

The present invention relates to the field of processes for manufacturing microcapsules of the reservoir-type microcapsule type, to the microcapsules thus prepared and to the use thereof in formulations such as washing products or cosmetic products.

The microcapsules known as reservoir-type microcapsules (also known as core/shell microcapsules) are microcapsules of the type containing an active principle in a polymer-based shell.

The processes for manufacturing these microcapsules, and thus for incorporating a lipophilic active principle into a polymer, comprise the steps consisting in:
dispersing at least one lipophilic active principle in an aqueous continuous phase, so as to form an emulsion or a dispersion of droplets of oil-in-water type,
polymerizing in situ a precursor of the polymer at the periphery of said droplets to form the wall of the shell of the microcapsules, enclosing the active principle.

### PRIOR ART

This microencapsulation may be performed according to two main routes:
One of the first routes involves a complex coacervation process using polymers of natural origin such as gelatin, as described in patent EP 0 674 942 B1. Said patent more specifically relates to the microencapsulation of hydrophobic chromogenic substances, the microcapsules being intended to be introduced into a coating composition for pressure-sensitive paper. Such microcapsules are not suitable for withstanding the presence of surfactants, such as in detergent medium.
A second route concerns processes involving the formation of synthetic copolymers, among which mention may be made of the polymerization of organic amine monomers or oligomers in the presence of aldehyde(s), notably using melamine/formaldehyde resins or using monomers such as silicates or silicones to make the shell of the microcapsules, which lead to microcapsules that are leaktight but which are not resistant in the presence of detergents. To overcome these drawbacks, patent EP 3 092 069 B1 has more recently disclosed a process for manufacturing microcapsules containing a lipophilic active agent, the double-walled shell of which is formed from two polymers, one being a silicone copolymer and the other an organic amine polymer, which is resistant to surfactants.

### AIMS OF THE INVENTION

The Applicant wished to at least partially dispense with the use of synthetic polymers, to improve the biodegradability of the microcapsules, while at the same time conserving their leaktightness and their capacity for resistance in the presence of surfactants.

A first aim of the invention is thus to propose a process for manufacturing reservoir-type microcapsules incorporating a lipophilic active agent using raw materials of natural origin, such as substances of animal or plant origin.

Another aim of the invention is to propose a process for manufacturing leaktight reservoir-type microcapsules that are resistant to surfactants, for the purpose of using them in detergent or cosmetic compositions.

Another aim of the invention is to propose a process for manufacturing reservoir-type microcapsules having a zero content of formaldehyde.

### SUMMARY OF THE INVENTION

To this end, the present invention relates to a process for manufacturing reservoir-type microcapsules, containing a lipophilic active principle in a shell forming the wall of said microcapsules.

According to the invention the process comprises the following steps:
(i) addition to a lipophilic phase, containing at least one active principle, of at least one silane and/or silicate monomer or oligomer, at least one melamine resin and at least one isocyanate, to form a mixture A,
(ii) dispersion with stirring of the mixture A obtained in step (i) in an aqueous continuous phase B at acidic pH containing at least one gelatin and/or at least one water-soluble plant protein, and at least one polyacid, so as to form, by complex coacervation, an emulsion or a dispersion of droplets of oil-in-water type, enveloped by a coacervate, and to initiate the formation of a first silicone/melamine/polyurethane copolymer enclosing said active principle,
(iii) optional introduction of a protective colloid such as a cellulose derivative into the aqueous continuous phase containing the dispersion of droplets,
(iv) addition, to the dispersion of droplets, at a temperature of less than or equal to 10° C., of at least one coacervate-crosslinking agent, enabling the co-crosslinking, at the interface of said droplets, of the gelatin-based and/or plant protein-based coacervate with the first silicone/melamine/polyurethane copolymer undergoing formation, leading to co-crosslinked polymers constituting the wall of said microcapsules and enclosing said active principle,
(v) production of an aqueous suspension of microcapsules enclosing the lipophilic active principle.

The polyacid of the aqueous phase B advantageously comprises a polyacid of the poly(meth)acrylic or polyaspartic type and a carboxyalkylcellulose, preferably carboxymethylcellulose.

The coacervate is the complex then formed by the association of the gelatin or protein which is positively charged in acidic medium with the negatively charged poly(meth)acrylic acid or polyaspartic acid and carboxymethylcellulose.

The main advantages of the process according to the present invention are that it enables the manufacture of “reservoir-type” microcapsules from inexpensive monomers that are readily available as regards the silicone/melamine/polyurethane copolymer and that it uses a biodegradable organic polymer (based on gelatin and/or plant protein).

The interest of this novel encapsulation process is also to combine the properties of highly crosslinked silicone membranes with those of organic membranes so as to obtain a custom barrier effect, and also to ensure good mechanical performance for the entire microcapsule structure by virtue of the covalent chemical reactions bonding the two types of polymers.

Thus, the shell forming the wall of said microcapsules is both biodegradable and resistant to surfactants.

### DETAILED DESCRIPTION OF THE INVENTION

Advantageously, the weight proportions of the constituents intended to form the silicone/melamine/polyurethane copolymer, introduced into the mixture A, are, respectively, from 50% to 80% of the silane and/or silicate monomer or oligomer, from 25% to 10% of the melamine resin and from 25% to 10% of the isocyanate, expressed as dry weight relative to the total weight of said constituents.

The aqueous phase B preferably contains the following weight proportions: 50% to 80% of gelatin and/or plant protein, 10% to 30% carboxyalkylcellulose, and 5% to 20% poly(meth)acrylic or polyaspartic type polyacid, expressed as a percentage of the dry weight of coacervate.

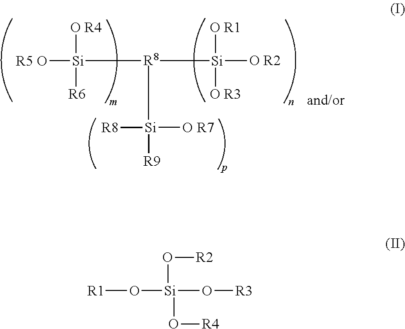

in which R1, R2, R3, R4, R5, R6, R7, R8 and R9 are substituted or unsubstituted, linear or cyclic alkyl radicals,
R° is an organic and/or silicone molecule,
the groups between ( ) being linked to R° via a silicon atom and are present m, n, or p times, m, n, p possibly being individually zero, but the sum m+n+p being at least equal to 1.

The value m+n+p, which is at least 1, is not limited theoretically, but practically by the viscosity of the product. The highly crosslinked silicone polymer used herein provides its hydrophobicity and reinforces the wall structure of the microcapsules.

The silane monomer or oligomer may be chosen, for example, from the compounds below:
the trialkoxysilanes of formula (I) in which m and p are zero: R—Si (OR1)(OR2)(OR3) in which R represents a substituted or unsubstituted alkyl radical containing 1 to 20 carbon atoms chosen from the following groups: methyl, ethyl, n-propyl, isopropyl, 1-n-butyl, 2-n-butyl, isobutyl, tert-butyl, n-pentyl, isopentyl, neopentyl, tert-pentyl, hexyl, heptyl, octyl such as n-octyl or isooctyl, 2,2,4-trim ethylpentyl, nonyl, decyl, dodecyl, octadecyl, cycloalkyl such as cyclopentyl, cyclohexyl and cycloheptyl and methylcyclohexyl, aryl such as phenyl, naphthyl, anthryl and phenanthryl, alkaryl such as o-, m- and p-tolyl, xylyl and ethylphenyl, and aralkyl such as benzyl, α- and β-phenylethyl,
R may also be halogenated, such as 3,3,3-trifluoro-n-propyl, 2,2,2,2′,2′,2′-hexafluoroisopropyl, heptafluoroisopropyl, o-, m- and p-chlorophenyl.

R may be unsaturated such as vinyl, 5-hexenyl, 2,4-divinylcyclohexylethyl, 2-propenyl, allyl, 3-butenyl and 4-pentenyl, ethynyl, propargyl and 2-propynyl.

R1, R2 and R3, which may be identical or different, are chosen, for example, from methyl and ethyl radicals, or may be oxygenated such as methoxyethyl, ethoxyethyl, acetoxy or oxymino.

A few advantageous monomers are mentioned as nonlimiting examples, among which are:
chloropropylmethyldimethoxysilane, γ-mercaptopropyltrimethoxy or ethoxy silane, γ-isocyanate propyltriethoxysilane,
epoxides: glycidoxypropyl trimethoxy or triethoxy silane, glycidoxy propylmethyldiethoxysilane, (3,4-epoxycyclohexyl)ethyltrim ethoxy or triethoxy silane,
acrylic silanes: acryloxypropyltrimethoxysilane, methacryloxypropyl)trim ethoxysilane, γ-methacryloxypropylmethyldimethoxy or diethoxy silane,
silanes bearing sulfur atoms: γ-mercaptopropyltrimethoxysilane, mercaptopropylmethyldimethoxysilane, bis{3-(triethoxysilyl)propyl} polysulfide, bis{3-(triethoxysilyl)propyl} disulfide, 3-octanoylthio-1-propyltriethoxysilane. The reason for this is that silanes bearing thiol groups react readily with isocyanate groups, leading to polythiourethanes which are of obvious interest here.
amino silanes: 3-aminopropyltriethoxy or methoxysilane, N-(n-butyl)-3-aminopropyltrimethoxy or ethoxy silane, N-aminoethyl-3-aminopropylmethyldimethoxysilane, N-aminoethyl-3-aminopropyltrimethoxy or triethoxysilane, 3-aminopropylmethyldiethoxysilane, N-phenylaminopropyl trimethoxysilane, 2-aminoethylaminopropyltrimethoxysilane, 2-aminoethylaminopropylmethyldimethoxysilane, anilinopropyltrimethoxysilane γ[N-(β-aminoethyl)amino]propylmethyldimethoxysilane, 4-amino-3,3-dimethylbutyltrimethoxysilane, 4-amino-3,3-dimethylbutylmethyldimethoxysilane, bis{γ-(trimethoxysilyl)propyl}amine, N-ethyl-γ-aminoisobutyl trimethoxysilane, 3-ureidopropyltriethoxysilane, hexamethyldisilazane, alkylene oxide trimethoxysilane, tris{3-(trimethoxysilyl)propyl} isocyanurate, bis(triethoxysilyl)ethane,
monoalkoxysilane or dialkoxysilane monomers intended for reducing the degree of crosslinking and thus making the silicone polymer more flexible.
monomers and prepolymers of the silicic ester type Si(OR′)₄, in which R′ is identical to the groups R′ described previously.

Needless to say, it is possible to use more complex monomers such as tris alkoxy isocyanurates or bis alkoxy isocyanurates, for example, and also oligomers of the products presented above.

Chlorinated silanes of the type R—Si—Cl₃, R—Si—Cl₂R′ or R—Si—ClR′R″ may also be used.

Advantageously, the compound of formula (II) is chosen from methyl polysilicate, ethyl polysilicate or a mixture thereof.

The melamine is preferably chosen from a liposoluble melamine-aldehyde or melamine-carbamate, and is preferably a melamine-carbamate. The melamine-carbamate resin is a resin which acts like melamine-formaldehyde resins while at the same time notably offering zero content of formaldehyde and good solubility in fragrances. It should be noted that this resin reacts with OH and other groups present in the coacervate and thus reinforces the structure. This is advantageous in the process of the present invention, since the presence of a melamine resin in the water would disrupt the entire system. Surprisingly, butanol or a different alcohol used to form and dissolve the melamine resin, which is released during the reaction of this polymer with the others, does not disrupt the correct progress of the complex coacervation.

The isocyanate used in mixture A of step (i) of the process according to the invention is preferably chosen from: toluene diisocyanate TDI, hexamethylene diisocyanate HDI, diphenylmethylene diisocyanate MDI, or isophorone diisocyanate IPDI, hexamethylene diisocyanate dimers or trimers, such as hexamethylene isocyanurate, uretdione, or uretonimine, or several thereof. However, HDI derivatives are preferred for their UV resistance and better hydrophilicity. Among the HDI derivatives, isocyanurate is advantageous on account of its low viscosity, its absence of volatility and also its solubility. Its reactivity is also appropriate. It should be noted that isocyanates also react with the OH, NH and NH₂ and also SH groups present in the coacervate and thus reinforce the wall structure.

The Applicant has also found that the isocyanate groups and the melamine resin make it possible to react with all the reactive groups present and thus to crosslink the entire structure. This has the advantage of considerably improving the strength of the microcapsules when compared with those obtained by complex coacervation only in washing products at temperatures in the region of 40° C.

It is noteworthy, and entirely surprising to a person skilled in the art, that none of the products used herein disrupt the complex coacervation, which is a rather delicate operation.

Step (ii) of the process is advantageously performed at a pH of between 3.0 and 5.5, preferably between 3.5 and 4.5, by adding to the aqueous phase at least one acid comprising nitric acid. Surprisingly, nitric acid, which is very rarely used in conventional complex coacervation, proved to be a good polymerization catalyst, complying with the above pH ranges, for the silicone/melamine/polyurethane copolymer and a good coacervate-forming agent. As a variant, hydrochloric acid, acetic acid, sulfuric acid, oxalic acid or formic acid may also be used.

According to a variant of the process of the present invention, step (ii) may be performed in two stages:
in a first stage, dispersion of the mixture A obtained in step (i) in an aqueous continuous phase B at acidic pH containing at least one gelatin and/or at least one water-soluble plant protein, at least one polyacid of poly(meth)acrylic type,
and then, in a second stage, addition of an aqueous solution of carboxyalkylcellulose to the dispersion obtained in the first stage.

The origin of the gelatin is not fundamental. It may be a pigskin gelatin, fish gelatin or other. It is even possible to replace all or some of this gelatin with a plant protein chosen for its water solubility. This last point is important for the cosmetic and pharmaceutical industry markets, for which products of animal origin are very poorly tolerated. Wheat, soybean or other cereal proteins or hydrolyzates of these plants may be used here, for example.

The binding and crosslinking agent introduced in step (iv) comprises glutaraldehyde.

Advantageously, the temperature ranges for the various steps of the process for manufacturing the microcapsules are as follows: step (i) is performed at room temperature (15-25° C.), step (ii) at a temperature of between 40° C. and 50° C., the emulsion formed then being cooled to a temperature of between 7° C. and 10° C., the glutaraldehyde is then added and this temperature is maintained for at least 4 hours, before completing the hot polymerization between 40° C. and 80° C. for 1 to 6 hours.

To summarize the steps of the process according to the invention, the “inner” copolymer is first prepared: the ingredients which are to form the silicone/melamine/polyurethane polymer, i.e. the inner polymer, are dissolved without heating (at room temperature) in the fragrance or other lipophilic inner phase by simple stirring to form the mixture A. The silicone precursor(s), the melamine resin(s) and the isocyanate(s) are thus successively introduced. Preferably, the less soluble and/or less reactive molecules are dissolved first, ending with the more reactive ones.

Once the internal phase is ready, the actual encapsulation is then performed rapidly to avoid premature polymerization reactions of the polymer coming from the internal phase.

The mixture A is then dispersed in water containing the polymers intended for the complex coacervation.

According to a first variant of the invention, the gelatin or the plant protein is dissolved in water, to which are added the polyacid of the poly(meth)acrylic or polyaspartic type and the pH-lowering nitric acid. The organic solution prepared beforehand, forming the mixture A, is then introduced into the aqueous mixture and emulsified, in the presence or absence of protective colloid (this protective colloid preferably being nonionized), to form by complex coacervation a polymer which becomes deposited around the droplets, thus producing an emulsion or a dispersion of oil-in-water type. The whole is then poured into a previously prepared carboxyalkylcellulose solution.

According to a second variant of the invention, the gelatin or plant protein is dissolved in water, to which are added the poly(meth)acrylic or polyaspartic type polyacid and the carboxyalkylcellulose. The organic solution prepared beforehand forming the mixture A is then introduced into the aqueous mixture and emulsified, in the presence or absence of preferably nonionized protective colloid. To initiate the complex coacervation, the pH is lowered, by adding acid, from about 6 to about 4.5, the coacervate then being deposited on the droplets and thus leading to an emulsion or dispersion of oil-in-water type.

The emulsion prepared at a temperature not exceeding 50° C. is subsequently cooled to about 8° C., and the glutaraldehyde is introduced optionally with other crosslinking agents. The whole is left without heating for several hours before raising the temperature and finishing the polymerizing for several hours at a higher temperature.

Finally, the operations are completed by returning to room temperature. An aqueous suspension of microcapsules is thus obtained.

The formation of the emulsion and the maintenance of its integrity during the encapsulation is promoted by the introduction of a water-soluble polymer into the continuous aqueous phase, known as a protective colloid. These products, which are well known to practitioners, may be, for example, cellulose derivatives such as hydroxyethylcellulose, methylcellulose, polyvinylpyrrolidone and polyvinylpyrrolidone copolymers, polyvinyl alcohols that are more or less hydrolyzed and also copolymers thereof, polymers of natural origin such as xanthan gum, alginates, pectins, starches and derivatives, casein, avoiding excessively ionized polymers which are liable to disrupt the complex coacervation.

Various metallic or organometallic catalysts may be used to complete the polymerization reaction. These may be, for example, tin-based compounds such as dibutyltin dilaurate or diacetate, tin octoate, inorganic tin salts, compounds of platinum, zinc, zirconium, aluminum, titanium including titanates, or fluorides, this list not being limiting.

The lipophilic active agents that may be encapsulated according to the process of the present invention are very numerous, the only limitation being that they withstand the temperature and pH conditions of the encapsulation steps and that they are sufficiently solvent to be capable of dissolving the reactive products introduced into the internal phase.

Among the advantageous active ingredients, mention will be made of fatty acids and alcohols, organic solvents, hydrocarbons, esters, silicone fluids and gums, plant oils and plant extracts, in particular products known for their cosmetic value, reactive or unreactive dyes and also pigment dispersions, UV-screening agents, vitamins and medically active molecules, fragrances, essential oils and flavorings, insecticides and repellents, catalysts, phase-change materials, phenolic compounds, and “color formers”.

The final aqueous suspension or dispersion of microcapsules generally contains from 30% to 40% by weight of active agent; it may be diluted, concentrated by the usual means, or even dried as a pulverulent powder.

The present invention also relates to the microcapsules manufactured via the process described above.

These reservoir-type microcapsules containing a lipophilic active principle, prepared by means of the above process, comprise a shell formed from at least two polymers bonded together by polar, hydrogen or covalent bonds forming the wall of said microcapsules, the first polymer, referred to as the internal polymer, being a silicone/melamine/urethane copolymer and the second polymer, referred to as the external polymer, being a crosslinked coacervate based on a gelatin polymer and/or plant protein, and a polyacid.

Advantageously, said external polymer represents between 15% and 65% by weight, preferably between 30% and 60% by weight, of the wall of said microcapsules. The fraction of material of natural origin of these microcapsules makes it possible to give them better biodegradability than the microcapsules of the prior art consisting exclusively of synthetic molecules.

The permeability of the microcapsules according to the invention may be modulated by modifying the conditions for polymerizing the wall, and also by modifying the dimensional features of the microcapsules, the diameter of which may range between 2 and 50 μm, preferentially between 5 and 20 micrometers.

Finally, it is advantageous to be able to vary the proportions of the biodegradable polymer used for the complex coacervation (external polymer) and also that for the internal synthetic polymer, since the two phases are prepared separately at the outset.

Thus, the greater the ratio of biodegradable polymer/synthetic polymer, the more biodegradable the microcapsules obtained will be and the more they will be able to be used, for example, in the cosmetics industry, the gelatin of animal origin then being replaced with a protein of plant origin.

Conversely, a lower ratio of biodegradable polymer/synthetic polymer will result in greater resistance of the microcapsules in the environments in which they are intended to be used.

Moreover, the weight proportion of wall/active principle of the microcapsules may vary within wide proportions, for example between 5% and 40%, preferentially between 7% and 25%.

The microcapsules according to the invention advantageously contain as active principle an odorous molecule, such as a fragrance.

The present invention also relates to the use of these microcapsules, notably in formulations containing surfactants.

More particularly, these microcapsules may be used in liquid washing products, washing powders, household and industrial detergents or fabric softeners.

In the cosmetic field, these microcapsules may be used in shampoos, hair-conditioning products, toothpastes, liquid soaps, body cleansers or lotions. The active principles may then be, for example, UV-screening agents, vitamins, unsaturated oils, or lipophilic active agents which may contain dyes or peptides.

The microcapsules prepared according to the process of the present invention may also be used in many other fields, such as the paper industry (NCR type carbonless copy paper, security papers), in the textile industry (cosmeto-textile, fragrances, phase-change materials, handkerchiefs, wipes), advertizing (fragranced advertisements, for example), the leather industry, the pharmaceutical industry, medicine, the veterinary industry, adhesives, paints and coatings, and construction, without this list being limiting.

### EXAMPLES

### Example 1: Microcapsules Containing a Fragrance, Prepared Via the Process According to the Present Invention

a) The following are successively introduced at room temperature (25° C.) into a 250 ml beaker magnetically stirred with a 45 mm bar:
140 g of mX floral fragrance from Iberchem
12.2 g of ethyl polysilicate (TES 40 from Wacker)
6.7 g of tris[3-(trimethoxysilyl)propyl] isocyanurate (Geniosil GF 69 from Wacker)
2.92 g of hexamethylene diisocyanate isocyanurate (HDT-LV2 tolonate from Perstorp)
5.2 g of melamine carbamate resin (Cymel 2000A from Allnex)
2.9 g of 3-mercaptopropyltrimethoxysilane (JH-S189 from JHSi).

b) A solution is prepared separately in a 1 L jacketed reactor stirred with a 4-blade impeller 7 cm in diameter and heated to 50° C., as follows:
80 g of tap water at 50° C.
2.25 g of carboxymethylcellulose (Wallocel CT 35GA from Dow).

c), A 600 ml beaker stirred with a turbine 6.5 cm in diameter is placed in a water bath regulated at 43° C. The following are successively introduced for dissolution:
141 g of tap water
0.33 g of hydroxyethylcellulose (250 m from Aqualon)
7.0 g of 140 bloom gelatin (PBG03 from Tessenderlo)
1.67 g of 20% nitric acid
2.8 g of sodium salt of a copolymer of acrylic and methacrylic acid (Synthran 8521 from Interpolymer) previously set at pH 4.5 with 50% sodium hydroxide.

[d) The solution in the fragrance prepared at the start is then poured into this beaker and emulsified at 42° C. for 30 minutes, the stirrer speed being regulated to obtain a mean diameter of 10 to 12 μm (speed between 1000 and 1400 rpm).

e) The contents of the beaker are then poured into the reactor containing the carboxymethylcellulose solution and the whole is cooled to 8° C. over 2 hours 30 minutes, the speed of said reactor being subsequently increased to avoid gelling which may possibly form on the edges of the reactor. 4.0 g of glutaraldehyde at 50% in water are then added. The temperature is maintained at 8° C. for a further 7 hours.

f) The reactor is then heated at 50° C. for 3 hours.

g) Finally, the reactor is returned to room temperature and the pH is adjusted to 5.5 with sodium hydroxide. Thickeners, preserving agents, deposition agents etc. are subsequently added.

### Example 2 (Comparative): Microcapsules Obtained by Standard Complex Coacervation

The preceding operations are repeated identically, but the fragrance of solution a) of the preceding example 1 is used alone, without any other product dissolved beforehand.

### Comparison of the Results

The microcapsules of examples 1 and 2 are compared in a fabric softener.

The features of the microcapsules prepared are collated in table 1 below:

TABLE 1

| | Internal | Mean | Formaldehyde |
|---|---|---|---|
| Type | reference | diameter | ppm |
| Example 2 | 9027 | 13.5 μm | 0 |
| Example 1 | 9087 | 11.3 μm | 0 |

### Procedure

Microcapsules containing 35% by weight of fragrance are incorporated into the standard commercial unfragranced fabric softener in a weight ratio of 2%, and mixed using a stirrer with vigorous stirring for 15 minutes.

Each of the mixtures is observed with the naked eye and then under a microscope and its stability is monitored over time.

### Results

After microscopic observation and photography, the observations relating to the various mixtures, immediately after incorporation into the washing product, are collated in table 2 below:

TABLE 2

| Microcapsules | In the fabric softener |
|---|---|
| Example 1 | Fairly well dispersed |
| Example 2 | Fairly well dispersed but deformed |
| (comparative) | |

10 days after accelerated aging at 50° C., the mixtures are observed again and the olfactory intensity is evaluated: the greater the intensity, the more the microcapsules have suffered (see table 3 below):

TABLE 3

| | | | | Intensity of the |
|---|---|---|---|---|
| Micro- | Appearance of the | Observation under | Fragrance intensity | fragrance on the |
| capsules | fabric softener | the microscope | in the softener | rubbed fabric |
| Example 1 | Beige-white color, | Correct dispersion | Very faint odor | Good odor, quite |
| | very homogeneous | | | strong |
| Example 2 | Thick | Fairly well dispersed | Very strong odor | Very faint odor |
| (comparative) | Beige color | But very deformed | | |

### CONCLUSION

The double-walled microcapsules are the most leaktight, they release less fragrance than the other microcapsules since they have suffered less attack by the surfactants present in the fabric softener.

The microcapsules obtained solely by complex coacervation are the ones that release the most odor into the fabric softener and which have thus become the most porous, which is confirmed by the very faint odor of the fabrics rubbed after 10 days.

1. A process for manufacturing reservoir-type microcapsules, containing a lipophilic active principle in a shell based on at least two covalently bonded polymers forming the wall of said microcapsules, the process comprising the following steps:
(i) addition to a lipophilic phase, containing at least one active principle, of at least one silane and/or silicate monomer or oligomer, at least one melamine resin and at least one isocyanate, to form a mixture A,
(ii) dispersion with stirring of the mixture A obtained in step (i) in an aqueous continuous phase B at acidic pH containing at least one gelatin and/or at least one water-soluble plant protein, and at least one polyacid, so as to form, by complex coacervation, an emulsion or a dispersion of droplets of oil-in-water type, enveloped by a coacervate, and to initiate the formation of a first silicone/melamine/polyurethane copolymer enclosing said active principle,
(iii) optional introduction of a protective colloid such as a cellulose derivative into the aqueous continuous phase containing the dispersion of droplets,
(iv) addition, to the dispersion of droplets, at a temperature of less than or equal to 10° C., of at least one coacervate-crosslinking agent, enabling the co-crosslinking, at the interface of said droplets, of the gelatin-based and/or plant protein-based coacervate with the first silicone/melamine/polyurethane copolymer undergoing formation, leading to co-crosslinked polymers constituting the wall of said microcapsules and enclosing said active principle,
(v) production of an aqueous suspension of microcapsules enclosing the lipophilic active principle.

2. The process as claimed in claim 1, wherein the polyacid of the aqueous phase B comprises a polyacid of poly(meth)acrylic or polyaspartic type and a carboxyalkylcellulose, preferably carboxymethylcellulose.

3. The process as claimed in claim 1, wherein the weight proportions of the constituents intended to form the silicone/melamine/polyurethane copolymer, introduced into the mixture A, are, respectively, from 50% to 80% of the silane and/or silicate monomer or oligomer, from 25% to 10% of the melamine resin and from 25% to 10% of the isocyanate, expressed as dry weight relative to the total weight of said constituents.

4. The process as claimed in claim 2, wherein the aqueous phase B contains the following weight proportions: 50% to 80% of gelatin and/or plant protein, 10% to 30% of carboxyalkylcellulose, and 5% to 20% of polyacid of poly(meth)acrylic or polyaspartic type, expressed as a percentage of the dry weight of coacervate.

5. The process as claimed in claim 1, wherein the silane compound(s) are chosen from the compounds of formula (I) or (II) below: 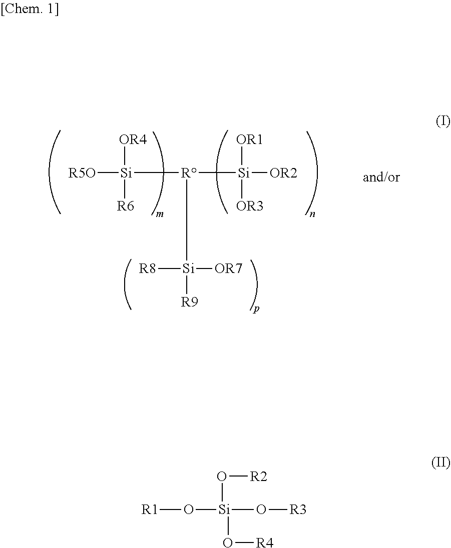
in which R1, R2, R3, R4, R5, R6, R7, R8 and R9 are substituted or unsubstituted, linear or cyclic alkyl radicals,
R° is an organic and/or silicone molecule,
The groups between ( ) being linked to R° via a silicon atom and are present m, n or p times, and m, n, p possibly being individually zero, but the sum m+n+p being at least equal to 1.

6. The process as claimed in claim 5, wherein the compound of formula (II) is chosen from methyl polysilicate, ethyl polysilicate or a mixture thereof.

7. The process as claimed in claim 1, wherein the melamine is chosen from a liposoluble melamine-aldehyde or melamine-carbamate, and is preferably a melamine-carbamate.

8. The process as claimed in claim 1, wherein the isocyanate is chosen from: toluene diisocyanate TDI, hexamethylene diisocyanate HDI, diphenylmethylene diisocyanate MDI, or isophorone diisocyanate IPDI, hexamethylene diisocyanate dimers or trimers, such as hexamethylene isocyanurate, uretdione, or uretonimine, or several thereof.

9. The process as claimed in claim 1, wherein step (ii) is performed at a pH of between 3.0 and 5.5, preferably between 3.5 and 4.5, by adding to the aqueous phase at least one acid comprising nitric acid.

10. The process as claimed in claim 2, wherein step (ii) is performed in two stages:
in a first stage, dispersion of the mixture A obtained in step (i) in an aqueous continuous phase B at acidic pH containing at least one gelatin and/or at least one water-soluble plant protein, at least one polyacid of poly(meth)acrylic type,
and then, in a second stage, addition of an aqueous solution of carboxyalkylcellulose to the dispersion obtained in the first stage.

11. The process as claimed in claim 1, wherein the crosslinking agent introduced into step (iv) comprises glutaraldehyde.

12. The process as claimed in claim 11, wherein step (i) is performed at room temperature (15-25 ° C.), step (ii) at a temperature of between 40° C. and 50° C., the emulsion formed being subsequently cooled to a temperature of between 7° C. and 10° C., the glutaraldehyde is then added and this temperature is maintained for at least 4 hours, before completing the hot polymerization between 40° C. and 80° C. for 1 to 6 hours.

13. A reservoir-type microcapsule containing a lipophilic active principle, prepared by means of the process as claimed in claim 1, comprising:
a shell formed from at least two polymers bonded together by covalent bonds forming the wall of said microcapsules, the first polymer, referred to as the internal polymer, being a silicone/melamine/urethane copolymer and the second polymer, referred to as the external polymer, being a crosslinked coacervate based on a gelatin polymer and/or plant protein, and a polyacid.

14. The microcapsule as claimed in claim 13, wherein said outer polymer represents between 15% and 65% by weight and preferably between 30% and 60% by weight of the wall of said microcapsules.

15. The microcapsule as claimed in claim 13, containing, as active principle, an odorous molecule, such as a fragrance.

16. The microcapsule prepared by means of the process as claimed in claim 1, wherein said microcapsule is configured for formulations containing surfactants.

17. The microcapsule prepared by means of the process as claimed in claim 1, wherein said microcapsule is configured for liquid washing products, washing powders, household and industrial detergents or fabric softeners.

18. The microcapsule prepared by means of the process as claimed in claim 1, wherein said microcapsule is configured for shampoos, hair-conditioning products, toothpastes, liquid soaps, body cleansers or lotions.
